(19) United States
(12) Patent Application Publication
(10) Pub. No.: US 2003/0185767 A1
(43) Pub. Date: Oct. 2, 2003

(54) Dental compositions

(76) Inventors: Terence Henry Lilley, South Yorkshire (GB); Thalie Paulina De Graaf, Nottinghamshire (GB); Yue Hugh Guan, Nottinghamshire (GB)

Correspondence Address: ROTHWELL, FIGG, ERNST & MANBECK, P.C., 1425 K STREET, N.W., SUITE 800, WASHINGTON, DC, 20005, US

(21) Appl. No.: 10/333,685

(22) PCT Filed: Jul. 25, 2001
(86) PCT No.: PCT/EP01/08605

(51) Int. Cl. A61K 7/16; A61K 7/28

(52) U.S. Cl. 424/49; 424/50

(57) ABSTRACT

Dental compositions to inhibit the adherence and plaque and/or stains on teeth comprise one or more water-soluble block copolymers of polyoxyethylene/polyoxypropylene which have a structure with the polyoxypropylene block being sandwiched between two polyoxyethylene blocks together with an acidic poly-amino acid or an alkali metal salt thereof.

This invention discloses dental compositions which prevent bacteria, plaque and stains from adhering to teeth.

Dental plaque is a general term for the complex microbial community existing on the tooth surface, embedded in a matrix of polymers of bacterial and salivary origin. Plaque that becomes calcified is referred to as calculus. Plaque has been implicated as the cause of caries, gingivitis and periodontal disease.

The control of plaque is very important since it has been implicated as the main cause of dental diseases. The main approaches have been mechanical plaque removal e.g. tooth-brushing or flossing or the use of chemical anti-microbial agents in oral care products such as toothpaste and mouthwash. Whilst toothbrushing easily removes plaque this is only a short term measure as the plaque rapidly recolonises the tooth surfaces and indeed may not be entirely removed from the more inaccessible areas such as fissures, interproximal spaces or the gingival crevice.

So however efficiently the teeth are cleaned, plaque build up starts almost from cessation of brushing. Bacteria rarely come into contact with bare enamel. As soon as the tooth surface is cleaned, salivary glycoproteins are adsorbed onto the surface forming the acquired salivary pellicle. Large numbers of bacteria are found in saliva (up to 10⁸ CFU ml⁻¹) which, unless swallowed, are likely to come into contact with a tooth surface and can initiate colonisation of that surface.

Coccal bacterial species such as S. sanguis, S. oralis and S. mitis are adsorbed onto the pellicle coated enamel within about 2 hours after cleaning. Other pioneer species such as Actinomyces are also found but obligatory anaerobic bacterial species are rarely detected at this stage. These primary colonising populations multiply, forming micro-colonies which become embedded in bacterial extracellular slimes and polysaccharides together with additional layers of adsorbed salivary proteins and glycoproteins. Growth of individual micro-colonies eventually results in the development of a confluent film of micro-organisms. The growth rates of the bacteria are fastest during this early period with doubling times from 1-3 hours having been calculated. As the plaque develops into a biofilm, metabolism by the pioneer species creates conditions suitable for colonisation by species with more demanding atmospheric and nutritional requirements. Also additional nutrients become available and the diversity of microbial cells in the microflora increases both in terms of the morphological types and the numbers of species.

Any method of oral hygiene needs to inhibit this rapid build up of plaque. Plaque control using antimicrobial agents has been a successful approach for many years. There may, however, be consequences from long term unsupervised use of these types of materials in oral care products such as disruption of the ecology of the oral microflora which may lead to the opportunistic overgrowth of more pathogenic species or to the development of resistant strains of organisms. An alternative approach is to modify the tooth surface coating and to make this less likely to form a good substrate for the initial colonisation of organisms.

As well as plaque control, it is expected that a dental composition will keep teeth looking nice, in particular by preventing staining. Extrinsic dental staining results from food, drink, tobacco, chromogenic bacteria and airborne particles. The exact mechanism of tooth staining is still subject to further investigations. However, it is widely thought to be dependent on the affinities of the chromogenic compounds for either the tooth surface, acquired pellicle, plaque or calculus present on the tooth surface.

After cleaning, proteins from saliva rapidly become deposited on the enamel surface to form the acquired salivary pellicle. As with plaque build-up, this pellicle forms a good substrate for the binding of compounds that are either coloured or become coloured due to chemical reactions such as the Maillard reaction.

Attempts to form films on teeth to prevent staining and decay have been previously documented.

U.S. Pat. No. 5,032,387 discloses a portable pump which dispenses amounts of a composition as a spray. The product is formulated as a spray to allow use as frequently as necessary. The composition contains cleaning agents such as surfactants and coating agents such as polymers and waxes. The coating agents have no solubility in water. These form a film over the teeth and prevent the adherence of plaque.

U.S. Pat. No. 5,645,841 teaches oral rinses containing a dispersion of silicone in a surfactant. The silicone is insoluble in the surfactant, but when dispersed in water, forms a coating on surfaces of the mouth. These oral rinses give improved antiplaque and antigingivitis activity. This is achieved as the coating acts a reservoir for various actives used to treat such conditions. Because of this, less ethanol is needed in the oral rinse to solubilise said actives.

WO 9414405 discloses a dentifrice containing a silicone oil. The oil enhances the polishing effect and shine of the teeth and reduces the extent to which the surfaces of the teeth are abraded. The silicone oil forms a film over the teeth. Any pellicle layer formed over this film is much easier to remove than normal plaque.

EP-A-839516 discloses dentifrices containing fatty acid triglycerides particularly capric and caprylic triglycedrides to reduce the adhesion of bacteria and plaque to the tooth surface.

This prior art teaches the application of water-insoluble hydrophobic molecules to inhibit the adherence and formation of plaque. Hydrophobic molecules of this type are extremely difficult to formulate into acceptable products. Complex emulsion systems are often required to produce a well mixed, stable and usable formulation.

We have now found that the use of one or more water-soluble block copolymers of polyoxyethylene/polyoxypropylene which have a structure with the polyoxypropylene block being sandwiched between two polyoxyethylene blocks together with an acidic poly-amino acid or an alkali metal salt thereof inhibits the adherence and formation of plaque and/or stains on the teeth.

The EO/PO/EO block copolymers are known generically as poloxamers and are available commercially, for example from BASF Corporation under the trade name Pluronic. Examples of the block copolymers sold under this trade name are given below. The data given is the molecular weight (MW), the melting point in degrees Celsius (M.p.), the HLB value (HLB) and the percentage of ethylene oxide present in the copolymer by weight (% EO)

A first group of suitable poloxamers have melting points in the range 48 to 58° C. Examples of this first group of poloxamers are given below.

| | F68 | F77 | F87 | F88 | F98 | F127 |
|---|---|---|---|---|---|---|
| MW | 8400 | 6600 | 7700 | 11400 | 13000 | 12600 |
| M.p. | 52 | 48 | 49 | 54 | 58 | 56 |
| HLB | 29 | 25 | 24 | 28 | 28 | 22 |
| % EO | 80 | 70 | 70 | 80 | 80 | 70 |

A second group of suitable poloxamers have melting points in the range 30 to 35° C. Examples of this second group of poloxamers are given below

| | P84 | P85 | P103 | P104 | P105 | P123 |
|---|---|---|---|---|---|---|
| MW | 4200 | 4600 | 4950 | 5900 | 6500 | 5750 |
| M.p. | 34 | 34 | 30 | 32 | 35 | 31 |
| HLB | 14 | 16 | 9 | 13 | 15 | 8 |
| % EO | 40 | 50 | 30 | 40 | 50 | 30 |

Where a single poloxamer is used it may be selected from the first or second group above. Preferably the poloxamer is selected from the second group.

Where a mixture of two poloxamers is used they may be chosen from the first or second groups above. Preferable both poloxamers are chosen from the second group. The ratio of the amounts of the two poloxamers may be in the range 80:20 to 20:80, preferably 60:40 to 40:60, most preferably around 50:50.

The total amount of poloxamer present in the compositions of the present invention may be 0.1 to 30%, preferably 0.5 to 20%, most preferably 1 to 15% by weight of the total composition.

Suitable poly-amino acids comprise polypeptides containing only or predominantly aspartic or glutamic acid moieties chemically linked by either αβ- or ββ-peptide bonds. Preferably the poly-amino acid is a polyaspartate polymer having a molecular weight of up to 50000, preferably 2000-10000, more preferably 2000 to 5000. These poly-amino acids are preferably used as their sodium salts. These poly-amino acids or their alkali metal salts can be characterised by measurement of pl by titration with a suitable titrant (HCl or NaOH) at ambient temperature. The pl value should be below pH 7 preferably in the range 2-5.

Suitable poly-amino acids and their alkali metal salts are available from Donlar Corporation or Bayer.

Such poly-amino acids and alkali metal salts are present at 0.001 to 1%, preferably 0.005 to 0.5, most preferably 0.01 to 0.1% by weight of the total composition.

The dental composition may be formulated as a toothpaste, mouthrinse, toothgel, toothpowder, dental tablet or a dental gel and may be formulated in a manner known to those skilled in the art.

Such compositions may, as appropriate, contain conventional materials such as, for example, humectants, surfactants, gelling agents, abrasives, fluoride sources, desensitising agents, flavourings, colourings, sweeteners, preservatives, structuring agents, bactericides, anti-tartar agents, chelating agents, whitening agents, vitamins, anti-plaque agents and any other therapeutic actives.

Suitable abrasives include particulate cellulose, silica, alumina, insoluble metaphosphates, calcium carbonate, dicalcium phosphate (in dihydrate and anhydrous forms), calcium pyrophosphate, natural and synthetic clays, and particulate thermosetting polymerised resins selected from melamine-ureas, melamine-formaldehydes, urea-formaldehydes, melamine-urea-formaldehydes, cross-linked epoxides, melamines, phenolics and cross-linked polyesters.

Suitable silica abrasives include the hydrated silicas, particularly those available under the trade names ‘Sident’ from Degussa AG, ‘Zeodent’ from J M Huber Corporation, ‘Sorbosil’ from Crosfield UK and Tixosil from Rhodia. Suitably, the particulate cellulose is highly purified cellulose such as that available under the trade names ‘Elcema’ from Degussa AG.

Suitable humectants for use in dentifrice compositions include polyhydric alcohols such as xylitol, sorbitol, glycerol, propylene glycol and polyethylene glycols. Mixtures of glycerol and sorbitol or sorbitol and xylitol are particularly effective. A humectant helps to prevent dentifrice compositions from hardening on exposure to air, and may also provide a moist feel, smooth texture, flowability and a desirable sweetness in the mouth. Suitably, such humectants may comprise from about 0-85% preferably from about 0-60% by weight of the oral hygiene composition.

Suitable surfactants for use in dentifrices, mouthwashes etc. are usually water-soluble organic compounds and may be anionic, non-ionic, cationic or amphoteric species. The surfactant should preferably be reasonably stable and able to produce a foam in use.

Anionic surfactants include the water soluble salts of C10-C18 alkyl sulphates (e.g. sodium lauryl sulphates), water-soluble salts of C10-C18 ethoxylated alkyl sulphates, water-soluble salts of C10-C18 alkyl sarcosinates, the water soluble salts of sulfonated monoglycerides of C10-C18 fatty acids (e.g. sodium coconut monoglyceride sulfonates), alkyl aryl sulfonates (e.g. sodium dodecyl benzene sulfonate), sodium salts of the coconut fatty acid amide of N-methyltaurine and sodium salts of long chain olefin sulfonates (e.g. sodium C14-C16 olefin sulfonate).

Non-ionic surfactants suitable for use in oral compositions include the products of alkylene oxide groups with aliphatic or alkylaromatic species, and may be for example, polyethylene oxide condensates of alkyl phenols, ethylene oxide/ethylene diamine copolymers, ethylene oxide condensates of aliphatic alcohols, long chain tertiary amine oxides, long chain tertiary phosphine oxides, long chain dialkyl sulfoxides and mixtures thereof. Alternatives include ethoxylated sorbitan esters such as those available from ICI under the trade name ‘Tween’.

Cationic surfactants are generally quaternary ammonium compounds having at least one C8-C18 alkyl or aryl chain and include, for example, lauryl trimethylammonium chloride, cetyl trimethylammonium bromide, cetyl pyridinium chloride, di-isobutylphenoxyethoxyethyldimethylbenzylammonium chloride, coconut alkyl trimethylammonium nitrate and cetyl pyridinium fluoride.

Also useful are benzyl ammonium chloride, benzyl dimethylstearylammonium chloride, and tertiary amines having one C1-C18 hydrocarbon group and two (poly)oxyethylene groups.

Amphoteric surfactants may be aliphatic secondary and tertiary amines comprising aliphatic species which may be branched or unbranched, and in which one of the aliphatic species is a C8-C18 species and the other contains an anionic hydrophilic group, for example, sulfonate, carboxylate, sulphate, phosphonate or phosphate. Examples of quaternary ammonium compounds are the quaternized imidazole derivatives available under the trade name ‘Miranol’ from the Miranol Chemical Company. Other amphoteric surfactants that may be employed are fatty acid amido alkyl betaines where one alkyl group is commonly C10-C12 such as cocoamido propyl betaine, for example Tego Betain supplied by T H Goldschmidt.

Suitably the surfactant is included in an amount from 0-20%, preferably 0-10%, most preferably 0.5-3% by weight of the oral hygiene composition.

Structuring agents may be required in, for example, dentifrices and gums to provide desirable textural properties and ‘mouthfeel’. Suitable agents include natural gum binders such as gum tragacanth, xanthan gum, gum karaya and gum arabic, seaweed derivatives such as Irish moss and alginates, smectitie clays such as bentonite or hectorite, carboxyvinyl polymers and water-soluble cellulose derivatives such as hydroxyethyl cellulose and sodium carboxymethyl cellulose. Improved texture may also be achieved, for example, by including colloidal magnesium aluminium silicate. Suitably, the structuring agents is included in an amount from 0-5%, preferably 0-3% by weight of the oral hygiene composition.

Fluoride sources suitable for use in all oral hygiene compositions of the present invention include sodium fluoride, zinc fluoride, potassium fluoride, aluminium fluoride, lithium fluoride, sodium monofluorophosphate, stannous fluoride, ammonium fluoride, ammonium bifluoride and amine fluoride.

Preferably, the fluoride source is present in an amount sufficient to provide from about 50 ppm to about 4,000 ppm fluoride ions in the composition. Inclusion of a fluoride source is beneficial, since fluoride ions are known to become incorporated into the hydroxyapatite of tooth enamel, thereby increasing the resistance of the enamel to decay. Fluoride is also now thought to act locally on the tooth enamel, altering the remineralisation-demineralisation balance in the favour of remineralisation. Inclusion of a fluoride source is also desirable when a polyphosphate anti-calculus agent is included, in order to inhibit the enzymatic hydrolysis of such polyphosphates by salivary phosphatase enzymes.

Suitable desensitising agents include, for example, formaldehyde, potassium salts such as potassium nitrate, tripotassium citrate, potassium chloride, potassium bicarbonate and strontium salts such as strontium chloride (suitably as hexahydrate), strontium acetate (suitably as hemihydrate) and also dibasic sodium citrate.

Flavouring agents may be added to increase palatability and may include, for example, menthol, oils of peppermint, spearmint, wintergreen, sassafras and clove. Sweetening agents may also be used, and these include D-tryptophan, saccharin, aspartame, levulose, acesulfam, dihydrochalcones and sodium cyclamate.

Typically, such flavouring agents are included in amounts from 0-5%, preferably from 0-2% by weight of the oral hygiene composition. Colouring agents and pigments may be added to improve the visual appeal of the composition. Suitable colourants include dyes and pigments. A suitable and commonly used pigment is titanium dioxide, which provides a strong white colour.

Suitably, as described above, the compositions of the invention may include further antimicrobial agents as preservative, antibacterial and/or anti-plaque agents. Suitable antimicrobial agents include water-soluble sources of certain metal ions such as zinc, copper and silver such as zinc citrate and silver chloride, the bis-biguanides such as chlorhexidine, aliphatic amines, phenolics such as bromochlorophene and triclosan, salicylanilides and quaternary ammonium compounds such as cetyl pyridinium chloride. Optionally, the formulations may also contain enzymes that will disrupt the pellicle or interfere with bacterial intercellular polysaccharides. Examples would include proteases such as papain and bromelain or dextranases. Natural enzymatic biocidal systems such as a system comprising lactoperoxidase and glucose oxidase may also be employed.

The composition may additionally comprise one or more anti-calculus agents. Suitable anticalculus agents include zinc salts such as zinc citrate and zinc chloride, polyphosphates and pyrophosphates. Suitable pyrophosphates include the sodium and potassium pyrophosphates, preferably disodium pyrophosphate, dipotassium pyrophosphate, tetrasodium pyrophosphate and tetrapotassium pyrophosphate and mixtures thereof. A preferred source of pyrophosphate is a mixture of tetrasodium pyrophosphate and tetrapotassium pyrophosphate. Suitable polyphosphates include sodium tripolyphosphate.

The invention is illustrated by the following non-limiting examples.

### EXAMPLE 1

### Anti-Adherent Toothpaste with Polyamino Acid

| | % w/w |
|---|---|
| Sorbitol (70% soln) | 20.00 |
| Hydrated Silica abrasive | 5.00 |
| Hydrated Silica Thickener | 9.72 |
| Sodium Polyaspartate (Mw 2000) (40%) | 0.1 |
| Flavour | 0.91 |
| Poloxamer (Pluronic P123) | 5 |
| Poloxamer (Pluronic F77) | 5 |
| Sodium Monofluorophosphate | 0.80 |
| Sodium Saccharin | 0.26 |
| Titanium Dioxide | 0.5 |
| Sodium Carboxymethylcellulose | 0.8 |
| Sodium Lauryl Sulphate | 0.2 |
| Water | qs |

The poloxamers, sodium monofluorophosphate and sodium saccharin were dissolved in water and sorbitol and sodium polyaspartate added. Hydrated Silica thickener, hydrated silica abrasive, titanium dioxide, sodium carboxymethylcellulose and sodium lauryl sulphate were added and mixed under vacuum. Flavour was added and the bulk mixed under vacuum until homogeneous.

### EXAMPLE 2

### Anti-Adherent Toothpaste without Polyamino Acid

| | % w/w |
|---|---|
| Sorbitol (70% soln) | 20.00 |
| Hydrated Silica abrasive | 5.00 |
| Hydrated Silica Thickener | 9.72 |
| Flavour | 0.91 |
| Poloxamer (Pluronic P123) | 5 |
| Poloxamer (Pluronic F77) | 5 |
| Sodium Monofluorophosphate | 0.80 |
| Sodium Saccharin | 0.26 |
| Titanium Dioxide | 0.5 |
| Sodium Carboxymethylcellulose | 0.8 |
| Sodium Lauryl Sulphate | 0.2 |
| Water | qs |

The poloxamers, sodium monofluorophosphate and sodium saccharin were dissolved in water and sorbitol added. Hydrated silica thickener, hydrated silica abrasive, titanium dioxide, sodium carboxymethylcellulose and sodium lauryl sulphate were added and mixed under vacuum. Flavour was added and the bulk mixed under vacuum until homogeneous.

### EXAMPLE 3

### Anti-Adherent Toothpaste with Polyamino Acid and Antibacterial

| | % w/w |
|---|---|
| Sorbitol (70% soln) | 20.00 |
| Hydrated Silica abrasive | 5.00 |
| Hydrated Silica Thickener | 9.72 |
| Flavour | 0.91 |
| Poloxamer (Pluronic P123) | 5 |
| Poloxamer (Pluronic F77) | 5 |
| Sodium Polyaspartate (Mw 2000) (40%) | 0.1 |
| Triclosan | 0.3 |
| Sodium Monoflurophosphate | 0.80 |
| Sodium Saccharin | 0.26 |
| Titanium Dioxide | 0.5 |
| Sodium Carboxymethylcellulose | 0.8 |
| Sodium Lauryl Sulphate | 0.2 |
| Water | qs |

The poloxamers, sodium monofluorophosphate and sodium saccharin were dissolved in water and sorbitol and sodium polyaspartate added. Hydrated silica thickener, hydrated silica abrasive, titanium dioxide, sodium carboxymethylcellulose and sodium lauryl sulphate were added and mixed under vacuum. Triclosan was dissolved in flavour and the solution added to the bulk and mixed under vacuum until homogeneous.

### EXAMPLE 4

### Control Toothpaste (Base)

| | % w/w |
|---|---|
| Sorbitol (70% soln) | 20.00 |
| Hydrated Silica abrasive | 5.00 |
| Hydrated Silica Thickener | 9.72 |
| Flavour | 0.91 |
| Sodium Monoflurophosphate | 0.80 |
| Sodium Saccharin | 0.26 |
| Titanium Dioxide | 0.5 |
| Sodium Carboxymethylcellulose | 0.8 |
| Sodium Lauryl Sulphate | 0.2 |
| Water | qs |

Sodium monofluorophosphate and sodium saccharin were dissolved in water. Hydrated silica thickener, hydrated silica abrasive, titanium dioxide, sodium carboxymethylcellulose and sodium lauryl sulphate were added and mixed under vacuum. Flavour was added and the bulk mixed under vacuum until homogeneous.

The ability of the selected materials and the exemplified dentifrices to prevent the adherence of bacteria was measured in vitro using hydroxyapatite (HA) discs.

An inoculum containing bacterial cells was prepared by adding 1 ml pooled saliva from human volunteers to 500 ml artificial saliva and incubating aerobically at 37° C. overnight to 1×10⁸ cells/ml in an artificial saliva. The artificial saliva contained 0.1% lab-lemco powder, 0.2% yeast extract powder, 0.5% proteose peptone, 0.25% hog gastric mucin, 6.0 mM NaCl, 1.8 mM CaCl2 and 2.7 mM KCl. Prior to use, 125 μl 40% urea aqueous solution was added to each 100 ml of this mixture.

Sterile HA discs were incubated at 37° C. for 30 min in 15-ml artificial saliva. The discs were then transferred to 15-ml aqueous solution containing selected test material(s) at the desired concentration or 15-ml toothpaste slurry (diluted 1:3 in artificial saliva). They were then incubated at 37° C. for 30 min. Following this, the discs were immersed in the inoculum prepared as described above and incubated at 37° C. for 18h. Each disc was then taken out and drained onto filter paper to remove non-adhering planktonic cells. After this, the discs were transferred to 10-ml buffered peptone and the adhering biofilms were disrupted by 1-min vortexing. Total cell numbers were counted microscopically using a haemocytometer. The antiadherent potential was obtained by comparing the total microbial counts for the test material(s)/toothpastes against the total microbial counts for the controls. All the operations were performed aseptically.

TABLE 1
Results for test material after 18 hr incubation

| | | Difference compared |
|---|---|---|
| Material | Cell Count | to control (%) |
| Control (Artificial saliva) | 8.63 × 10⁷ | 0 |
| 5% Pluronic P123 + | 6.63 × 10⁷ | 23.2 |
| 5% Pluronic F77 | | |
| 0.1% Polyaspartate | 7.80 × 10⁷ | 9.6 |
| 5% Pluronic P123 + | 5.13 × 10⁷ | 40.6 |
| 5% Pluronic F77 + | | |
| 0.1% Polyaspartate | | |

TABLE 2
Bacterial adherence results for test materials after 18 h incubation

| | | Difference |
|---|---|---|
| | | compared to |
| Material(s) in solution or dispersion | Cell count | the control (%) |
| Control (Artificial saliva) | 2.12 × 10⁸ | 0 |
| 0.1% Polyaspartate | 1.23 × 10⁸ | 41.9 |
| 1% Polyaspartate | 1.39 × 10⁸ | 34.3 |
| 10% Pluronic P104 | 1.89 × 10⁸ | 10.6 |
| 10% Pluronic P104 + 0.1% Polyaspartate | 5.91 × 10⁷ | 72.1 |
| 10% Pluronic P104 + 1% Polyaspartate | 7.78 × 10⁷ | 63.3 |
| 10% Pluronic F88 | 1.33 × 10⁸ | 37.3 |
| 10% Pluronic F88 + 0.1% Polyaspartate | 3.82 × 10⁷ | 82.0 |
| 10% Pluronic F88 + 1% Polyaspartate | 7.59 × 10⁷ | 64.2 |
| 5% Pluronic P84 + 5% Pluronic P104 | 1.58 × 10⁸ | 25.4 |
| 5% Pluronic P84 + 5% Pluronic P104 + | 6.57 × 10⁷ | 69.0 |
| 0.1% Polyaspartate | | |
| 5% Pluronic P84 + 5% Pluronic P104 + 1% | 5.91 × 10⁷ | 72.1 |
| Polyaspartate | | |

TABLE 3
Results for Toothpastes after 18 hr incubation

| | | Difference |
|---|---|---|
| | | compare to |
| | Cell Count | control (9%) |
| Control toothpaste | 7.23 × 10⁷ | 0 |
| (Example 4) | | |
| Test Toothpaste | 5.13 × 10⁷ | 29 |
| (Example 1) | | |
| Test Toothpaste | 6.23 × 10⁷ | 14 |
| (Example 2) | | |

Statistical analysis of the results in Table 3 showed a significant difference between test and control demonstrating the increased efficacy of using EO/PO/EO block copolymers together with an acidic poly-amino acid or its alkali metal salt in preventing the adherence of plaque.

1. A dental composition to inhibit the adherence and formation of plaque and/or stains on teeth comprising two or more different water-soluble block copolymers of polyoxyethylene/polyoxypropylene which have a structure with the polyoxypropylene block being sandwiched between two polyoxyethylene blocks, together with an acidic poly-amino acid or an alkali metal salt thereof.

2. Dental composition as claimed in claim 1 wherein the composition comprises two water-soluble block copolymers of polyoxyethylene/polyoxypropylene.

3. Dental composition as claimed in claim 1 wherein at least one of the water-soluble block copolymers of polyoxyethylene/polyoxypropylene is selected from poloxamers having a melting point in the range 30 to 35° C.

4. Dental composition as claimed in claim 2 wherein both of the water-soluble block copolymers of polyoxyethylene/polyoxypropylene are selected from poloxamers having a melting point in the range 30 to 35° C.

5. Dental composition as claimed in claim 5 wherein the polyamino acid is a polypeptide containing only or predominantly aspartic and glutamic acid moieties linked by either aβ- or ββ-peptide bonds.

6. Dental composition as claimed in claim 5 wherein the polyamino acid is a polyaspartate polymer having a molecular weight of up to 50000.

7. Dental composition as claimed in claim 5 wherein the polyamino acid is a polyaspartate polymer having a molecular weight in the range 2000 to 10000.

8. Dental composition as claimed in claim 5 wherein the polyamino acid is a polyaspartate polymer having a molecular in the range 2000 to 5000.

9. Dental composition as claimed in any one of claims 5 to 8 wherein the polyaspartate polymer is present in the form of its sodium salt.

10. Dental composition as claimed in any preceding claim wherein the amount of copolymers of polyoxyethylene/polyoxypropylene is in the range 0.1% to 30% by weight of the total composition.

11. Dental composition as claimed in any preceding claim wherein the amount of copolymers of polyoxyethylene/polyoxypropylene is in the range 0.5% to 20% by weight of the total composition.

12. Dental composition as claimed in any preceding claim wherein the amount of copolymers of polyoxyethylene/polyoxypropylene is in the range 1% to 15% by weight of the total composition.

13. Dental composition as claimed in claim 2 wherein the ratio of the amounts of the two poloxamers is in the range 80:20 to 20:80.

14. Dental composition as claimed in claim 2 wherein the ratio of the amounts of the two poloxamers is in the range 60:40 to 40:60.

15. Dental composition as claimed in claim 2 wherein the ratio of the amounts of the two poloxamers is 50:50.

16. Dental composition as claimed in any preceding claim, formulated as a toothpaste, mouthrinse, toothgel, toothpowder, dental tablet or a dental gel.
